(19) United States
(12) Patent Application Publication
(10) Pub. No.: US 2013/0004380 A1
(43) Pub. Date: Jan. 3, 2013

(54) STERILIZING APPARATUS AND METHOD USING HYDROGEN PEROXIDE

(76) Inventor: Chul Won Yoo, Seoul (KR)

(21) Appl. No.: 13/635,462

(22) PCT Filed: Mar. 16, 2011
(86) PCT No.: PCT/KR11/01846

§ 371 Date: Sep. 17, 2012

(30) Foreign Application Priority Data
Mar. 17, 2010 (KR) 10-2010-0023704

(51) Int. Cl. A61L 2/20

(52) U.S. Cl. 422/186; 422/292

(57) ABSTRACT

Disclosed is a sterilization apparatus using hydrogen peroxide, which comprises a housing which has a sterilization chamber for accommodating to-be-sterilized objects; an evaporator connected with the sterilization chamber for supplying hydrogen peroxide vapor to the sterilization chamber for the purpose of sterilizing to-be-sterilized objects; a hydrogen peroxide supply apparatus connected with the evaporator for the purpose of supplying liquid hydrogen peroxide to the evaporator; a vacuum pump connected with the sterilization chamber for the purpose of providing a vacuum pressure to the sterilization chamber; a blower which includes a fan disposed in the interior of the sterilization chamber for forcibly flowing the air of the sterilization chamber and a motor for driving the fan; and a fan heater disposed in the interior of the sterilization chamber for the purpose heating the air flowing by means of the fan. The sterilization apparatus using hydrogen peroxide according to the present invention is directed to quickly raising the temperature of the to-be-sterilized objects in the interior of the sterilization chamber with the aid of the convection phenomenon of the heated wind using the fan and the fan heater.

### TECHNICAL FIELD

The present invention relates to an apparatus for sterilizing a to-be-sterilized object using a chemical sterilant, and particularly to a sterilization apparatus and method using a hydrogen peroxide which makes it possible to sterilize a to-be-sterilized object using a hydrogen peroxide.

### BACKGROUND ART

In the sterilization on medical tools, different from a cleaning or disinfection, it means that all kinds of living microorganisms are fully sterilized by way of physical, chemical operations, so the sterilization means a high level treatment. The medical tools are sterilized using high temperature dry heat or the heat from vapor or in such a way that chemicals such as formaldehyde or ethylene oxide are used in a gas or vapor state.

A plurality of different medical tools such as a fiber optical device, an endoscope, a certain motored tool, etc. are quite weak to heat or moisture or both of them, so the conventional sterilization method using heat cannot be actually adapted. The method of using formaldehyde or ethylene oxide gas is a toxic gas with potential cancer-causing factors, so a lot of attentions are needed when handling.

In particular, when ethylene oxide gas is used, an aeration time of more than 8 hours is needed so as to eliminate the gas from the sterilized products, which becomes a huge factor consequently prolonging sterilization cycle time. In terms of the installation environment and management, an independent space and the provision of a gas detector are needed, the work becomes complicated.

In addition to a sterilization method of using ethylene oxide gas of formaldehyde gas, there is a sterilization method of using plasma. In a conventional sterilization apparatus using plasma, when hydrogen peroxide is injected in a plasma state, the pressure rises, which makes it hard to generate plasma, and when plasma is generated in a state that hydrogen peroxide is injected, the plasma comes to decompose hydrogen peroxide. So, the plasma process is performed before or after the injection of the hydrogen peroxide, which means the sterilization by means of hydrogen peroxide rather than plasma.

In the conventional sterilization apparatus using plasma, the to-be-sterilized object with a complicated inner structure such as an endoscope is hard to be sterilized with a radiation heat by way of plasma. In addition, a vacuum environment is needed to generate plasma. When the moisture is vaporized under the vacuum state, the vacuum pressure lowers, so it is hard to generate plasma. As a result, the whole sterilization process becomes complicated, and the effects from the plasma worsen.

In terms of the sterilization method using hydrogen peroxide and plasma, it is true that the temperature of the sterilization chamber and the temperature of the to-be-sterilized object affect the sterilization efficiency; however in the conventional low temperature sterilizer using a temperature lower than 60° C., a low temperature plasma might be used in the vacuum state or a more passive method of using an internal radiation heat is used while maintaining the sterilization chamber to be lower than 60° C. in the outside. In a conventional way, the hospitals feature in that the to-be-sterilized objects are washed, and waters are removed, and then the sterilization is conducted using the sterilizer. The conventional plasma and hydrogen peroxide sterilization apparatus frequently causes incomplete sterilization, so re-sterilization processes are somehow needed.

The incomplete sterilization occurs because when the temperature of sterilization lowers in the sterilization chamber due to the to-be-sterilized objects, the vaporized hydrogen peroxide solution does not deeply penetrate the complicated to-be-sterilized objects having a spread limiting region such as long, narrow lumen and becomes condensed, so even though too much amount of hydrogen peroxide is injected, the vaporizing amount becomes quite limited when the temperature is low.

In the conventional sterilization method and sterilization apparatus using a low temperature plasma and hydrogen peroxide do not have dry functions. So, the sterilized objected are dried using a separate dryer, which causes many inconveniences, and the whole work time is prolonged.

Thanks to the above mentioned technical problems, a new method of enhancing the sterilization effects using hydrogen peroxide is needed, and a simpler, more effective method is needed for the sake of a perfect dry in terms of the low temperature plasma and hydrogen peroxide sterilizations.

### DISCLOSURE OF INVENTION

Accordingly, the present invention is made to improve the above mentioned problems, and it is an object of the present invention to provide a sterilization apparatus and method using hydrogen peroxide which make it possible to prevent incomplete sterilization in such a way to uniformly raise the temperatures of a sterilization chamber and a to-be-sterilized object and to diversify the processes for the purpose of adjusting a sterilization time and a sterilization degree matching with each to-be-sterilized object, thus greatly enhancing the sterilization efficiency with respect to the different items and the different to-be-sterilized objects each having narrow, long regions.

To achieve the above objects, there is provided a sterilization apparatus using hydrogen peroxide, comprising a housing which has a sterilization chamber for accommodating to-be-sterilized objects; an evaporator connected with the sterilization chamber for supplying hydrogen peroxide vapor to the sterilization chamber for the purpose of sterilizing to-be-sterilized objects; a hydrogen peroxide supply apparatus connected with the evaporator for the purpose of supplying liquid hydrogen peroxide to the evaporator; a vacuum pump connected with the sterilization chamber for the purpose of providing a vacuum pressure to the sterilization chamber; a blower which includes a fan disposed in the interior of the sterilization chamber for forcibly flowing the air of the sterilization chamber and a motor for driving the fan; and a fan heater disposed in the interior of the sterilization chamber for the purpose heating the air flowing by means of the fan.

The fan is disposed closer to a side wall of one side of the housing, and the motor is disposed at an outer side of the side wall, and a rotary shaft of the motor passes through the side wall and is connected with the fan.

The fan heater is disposed between the fan and the side wall.

At the fan is installed a guide plate which faces the fan at the front side of the fan in the interior of the sterilization chamber and which rotates in a rotation direction that the air flowing to the fan gathers at the center of the fan, and the air of the interior of the sterilization chamber collides with the side wall by way of the fan and the fan heater and flows to the front side of the fan by way of the space between the guide plate and the inner surface of the housing.

In the sterilization apparatus using hydrogen peroxide, there is further provided a sterilization chamber temperature sensor disposed at the passage for detecting the temperature of the interior of the sterilization chamber.

In the sterilization apparatus using hydrogen peroxide, there are further provided a photo catalyst disposed in the interior of the sterilization chamber; and a violet ray generator which has a violet ray lamp disposed in the interior of the sterilization chamber for emitting violet ray to the photo catalyst.

The photo catalyst is coated on the surface of the fan.

The photo catalyst contains TiO₂.

In the sterilization apparatus using hydrogen peroxide, there is further provided a sterilization chamber pressure release valve connected with the sterilization chamber for introducing external air into the interior of the sterilization chamber.

In the sterilization apparatus using hydrogen peroxide, there is further provided a sterilization chamber heater which is engaged to the housing for the purpose of heating the housing and raising the internal temperature of the sterilization chamber.

The sterilization method using hydrogen peroxide according to an embodiment of the present invention comprises (a) inputting a to-be-sterilized object into the interior of the sterilization chamber and supplying liquid hydrogen peroxide into an evaporator connected with the sterilization chamber, (b) raising the temperature of the to-be-sterilized object in such a way to operate a fan and a fan heater installed in the interior of the sterilization chamber, to heat the air in the interior of the sterilization chamber, and to forcibly flow it, (c) supplying the hydrogen peroxide vapor from the evaporator into the sterilization chamber by opening the flow passage connecting the sterilization chamber and the evaporator, and (d) heating the air in the interior of the sterilization chamber by operating the fan and the fan heater installed in the interior of the sterilization chamber and forcibly flowing it.

The sterilization method using hydrogen peroxide according to an embodiment of the present invention further comprises making the pressure of the sterilization chamber lower than atmospheric pressure before (c).

After (c), there is further provided a step of making the pressure of the sterilization chamber have atmospheric pressure by supplying external air to the sterilization chamber.

The sterilization method using hydrogen peroxide according to an embodiment of the present invention further comprises emitting violet ray to the photo catalyst disposed in the interior of the sterilization chamber before or after (c).

### Advantageous Effects

The sterilization apparatus using hydrogen peroxide according to the present invention is directed to quickly raising the temperatures of the to-be-sterilized objects in the interior of the sterilization chamber by means of the convention phenomenon of warm wind using a fan and a heater, and the temperature of the air in the sterilization chamber and the to-be-sterilized objects can be raised uniformly. So, when the hydrogen peroxide vaporizes, the moisture first penetrates into a long, narrow spread limit region, thus preventing the condensation, and the hydrogen peroxide does not condense, and more hydrogen peroxide can vaporize, thus enhancing the sterilization performance.

In addition, the sterilization apparatus using hydrogen peroxide according to the present invention is directed to converting the oxygen, moisture in the air and the injected hydrogen peroxide into plasma states, and they are circulated using the fan, thus producing high density OH radicals. So, expensive plasma can be produced at a low cost, and the sterilization performance can be enhanced more.

The sterilization apparatus using a hydrogen peroxide according to the present invention is directed to vacuuming after the air and the to-be-sterilized objects in the interior of the sterilization chamber are fully heated sing a heated air convection phenomenon generated with the aid of the fan and the heater, so the moisture can be quickly eliminated from the to-be-sterilized and the sterilization chamber, so the to-be-sterilized objects can be fully dried without using a separate drier. Since the separate drier is not needed in the present invention, the facility cost can be saved.

In addition, the sterilization apparatus using a hydrogen peroxide according to the present invention is directed to enhancing deodorization effects in such a manner to change the moisture contained in the air into OH radicals with the aid of the photo catalyst and ultraviolet ray.

### BRIEF DESCRIPTION OF DRAWINGS

FIG. 1 is a schematic view illustrating a construction of a sterilization apparatus using hydrogen peroxide according to an embodiment of the present invention.

FIG. 2 is a schematic block diagram illustrating a sterilization apparatus using hydrogen peroxide according to an embodiment of the present invention.

FIG. 3 is a graph of a comparison of the temperature changes in the sterilization chamber as time passes when a fan and a heat heater are not used and they are used.

FIG. 4 is a view illustrating a result of the hydrogen peroxide concentration in the sterilization chamber based on the temperature in the sterilization chamber and the concentration of hydrogen peroxide.

FIG. 5 is a graph showing a result of the measurement performed every 10 seconds with respect to the change in the concentration of hydrogen peroxide by comparing the results before and after the fan and the violet ray generator are operated.

### DESCRIPTIONS OF THE REFERENCE NUMERALS

10: sterilization apparatus
11: sterilization chamber
12: housing
13: evaporator
14: hydrogen peroxide supplier
15: vacuum pump
16: blower
17: violet ray generator
18: door
19: filter
20: hydrogen peroxide vapor supply adjusting valve
21: sterilization chamber pressure adjusting valve
22: sterilization chamber pressure release valve
23: sterilization chamber pressure meter
24: fan heater
25, 26: first and second sterilization chamber heaters
30: sterilization chamber temperature sensor
31: hydrogen peroxide supply adjusting valve
32: evaporator
34: fan
35: motor
36: photo catalyst
37: guide plate
39: violet ray lamp
41: controller

### BEST MODES FOR CARRYING OUT THE INVENTION

Hereinafter, the sterilization apparatus and method using hydrogen peroxide according to an embodiment of the present invention will be described.

During the descriptions of the present invention, the size and shape of the elements shown in the drawings might be seen over emphasized for a clear, convenient and simplified construction. In addition, the terms defined in consideration of the construction and operation of the present invention might differ from the operator's intention or practices. The terms should be interpreted as the meaning and concept matching with the technical concepts of the present invention based on the contents over the specifications of the present invention.

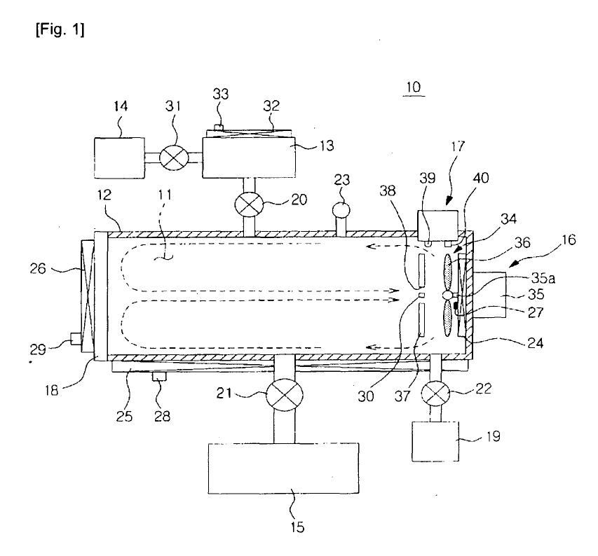

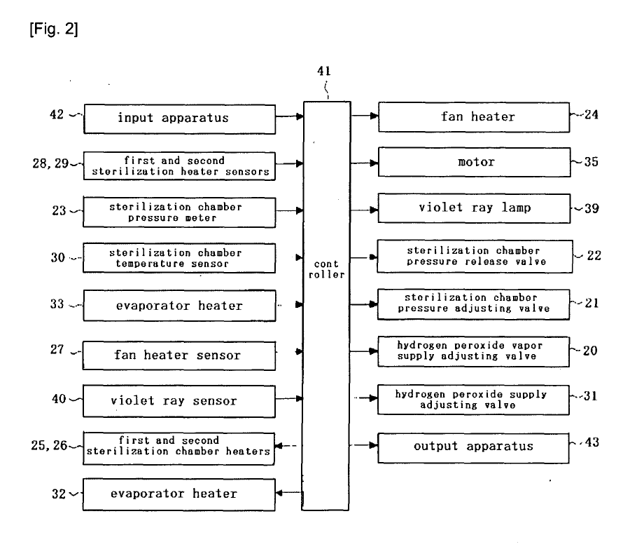

FIGS. 1 and 2 are schematic views illustrating a sterilization apparatus using hydrogen peroxide according to an embodiment of the present invention.

As shown in FIG. 1, the sterilization apparatus 10 using hydrogen peroxide according to an embodiment of the present invention comprises a housing 12 with a sterilization chamber 11 filled with a to-be-sterilized object, an evaporator 13 configured to supply hydrogen peroxide vapor to the sterilization chamber 11, a hydrogen peroxide supply apparatus 14 configured to supply hydrogen peroxide to the evaporator 13, a vacuum pump 15 configured to adjust the internal pressure of the sterilization chamber 11, a blower 16 configured to forcibly flow the air in the interior of the sterilization chamber 11, and a violet ray generator 17 configured to emit violet ray. At one side of the housing 12 is engaged a door 18 for opening and closing the opening which is used as a doorway for the sake of the to-be-sterilized objects.

The sterilization chamber 11 is connected with the evaporator 13, the vacuum pump 15 and the filter 19 by way of a plurality of tubes each with a flow passage for the fluid to flow. At the tube connecting the sterilization chamber 11 and the evaporator 13 are installed the hydrogen peroxide vapor supply adjusting valve 20, and at the tube connecting the sterilization chamber 11 and the vacuum pump 16 is installed the sterilization chamber pressure adjusting valve 21. In addition, at the tube connecting the sterilization chamber 11 and the filter 19 is installed a sterilization chamber pressure release valve 22. The hydrogen peroxide vapor supply adjusting valve 20, the sterilization chamber pressure adjusting valve 21 and the sterilization chamber pressure release valve 22 are controlled by means of the controller 41.

The hydrogen peroxide vapor supply adjusting valve 20 is configured to adjust the supply of the hydrogen peroxide vapor with respect to the sterilization chamber 11 as the passage connecting the sterilization chamber 11 and the evaporator 13 are closed and opened. The sterilization chamber pressure adjusting valve 21 is configured to open and close the flow passage between the sterilization chamber 11 and the vacuum pump 15. When the vacuum pump 15 starts operating in a state that the sterilization chamber pressure adjusting valve 21 is open, the air of the sterilization chamber 11 is discharged to the vacuum pump 15, and the inner pressure of the sterilization chamber 11 lowers. The sterilization chamber pressure release valve 22 is configured to introduce the external air into the interior of the sterilization chamber 11, and when the sterilization chamber pressure release valve 22 is open, the external air is introduced into the sterilization chamber 11, and the inner pressure of the sterilization chamber 11 rises.

The internal pressure of the sterilization chamber 11 is measured by means of the sterilization chamber pressure meter 23 installed in the housing 12, and the controller 41 receives a detection signal of the sterilization camber pressure meter 23 and controls the operations of the vacuum pump 15, the sterilization chamber pressure adjusting valve 21, the sterilization chamber pressure release valve 22, thus properly adjusting the internal pressure of the sterilization chamber 11. The filter 19 serves to prevent the contaminated air from coming into the interior of the sterilization chamber 11 when the sterilization chamber pressure release valve 22 is open.

The internal temperature of the sterilization chamber 11 is adjusted by the fan heater 24 installed in the interior of the sterilization chamber 11, the first sterilization chamber heater 25 installed in the housing 12, and the second sterilization camber heater 26 installed at the door 18. The heaters 24, 25 and 26 are controlled by means of the controller 41. The controller 41 serves to receive the signals from the first sterilization chamber heater sensor 28, the second sterilization chamber heater sensor 29 and the sterilization chamber temperature sensor 30 installed in the sterilization chamber 11 and controls the heaters 24, 25 and 26 for thereby constantly maintaining the temperature of the sterilization chamber 11.

The evaporator 13 is connected with the hydrogen peroxide supply apparatus 14 by way of a tube with a flow passage. At the tube connecting the evaporator 13 and the hydrogen peroxide supply apparatus 14 is installed a hydrogen peroxide supply adjusting valve 31 which is controlled by the controller 41. The hydrogen peroxide supply apparatus 14 serves to supply liquid hydrogen peroxide to the evaporator 13. The temperature of the evaporator 13 is adjusted by means of the evaporator heater 32 controlled by means of the controller 41. The controller 41 serves to constantly maintain the temperature of the evaporator 13 in such a way to control the evaporator heater 32.

As shown in FIG. 1, the blower 16 is formed of a fan 34 disposed in the interior of the sterilization chamber 11 and a motor 35 for the purpose of rotating the fan 34. The motor 35 is installed at an outer side of the housing 12, and the rotary shaft 35a of the motor 35 passes through the side wall of the housing 12 and is connected with the fan 34. As not shown, at a side wall of the housing 12 that the rotary shaft 35a of the motor 35 passes through is installed a sealing member for the purpose of preventing the communication with the sterilization chamber 11 by way of the flow of air. The surface of the fan 34 is coated with a photo catalyst 36 such as TiO₂.

The fan 34 is disposed close to the side wall of one side of the sterilization chamber 11, so the air in the sterilization chamber 11 can be more effectively circulated. A guide plate 37 is disposed at a front side of the fan 34 for the sake of smooth flow of air. The guide plate 37 has a passage 38 facing the fan 34. The fan 34 rotates in the rotation that the air of the sterilization camber 11 is pulled, and the air of the sterilization chamber 11 moves to the rear side of the fan 34 by way of the passage 38 of the guide plate 37 and the fan 34.

The air flowing to the rear side of the fan 34 is heated by means of the fan heater 24 disposed between the fan 34 and the side wall, and the air passed through the fan heater 24 collides with the side wall and flows to the outer side of the fan heater 24 along the side wall, and the air flowing to the fan heater 24 flows to the front side of the fan 34 by way of the space between the guide plate 37 and the upper surface of the sterilization chamber 11, the space between the guide plate 37 and the lower surface of the sterilization chamber 11 or the space between the guide plate 37 and the front and rear walls of the sterilization chamber 11. So, the whole air of the sterilization chamber 11 can be quickly heated. The sterilization chamber temperature sensor 30 configured to detect the internal temperature of the sterilization chamber 11 is arranged at the passage 38 of the guide plate 37.

In the present invention, the air of the sterilization chamber 11 can be heated using either the first sterilization chamber heater 25 or the second sterilization chamber heater 26 surrounding the sterilization chamber 11 without using the fan heater 24. In addition, the rotation direction of the fan 34 might be opposite to the above described direction.

As shown in FIG. 1, at one side of the sterilization chamber 11 is installed a violet ray generator 17. The violet ray generator 17 consists of a violet ray lamp 39 generating violet ray and a violet ray sensor 40 for detecting the violet ray. Here the violet ray lamp 39 and the violet ray sensor 40 are controlled by the controller 41. The controller 41 serves to control the operations of the violet ray lamp 39 in response to a signal from the violet ray sensor 40. The moisture contained in the air can be converted into OH radicals using the photo catalyst 36 and the violet ray, thus enhancing the immunity along with the enhanced deodorizing effects. The detailed operations of the photo catalyst 36 and the violet ray will be described later. The photo catalyst 36 might be provided on the inner surface of the housing 12 in addition to the fan 34 or the guide plate 37 in the interior of the sterilization chamber 11.

As shown in FIG. 2, the controller 41 serves to control the whole operations of the sterilization apparatus 10 by receiving a control signal from various sensors and pressure meter and the input apparatus 42. The controller can output the operation states of the sterilization apparatus 10, such as the temperature and pressure of the chamber 11 with the aid of the output apparatus 43.

The procedures of sterilizing the to-be-sterilized objects using the sterilization apparatus using hydrogen peroxide according to an embodiment of the present invention will be described with reference to the accompanying drawings.

The to-be-sterilized objects are washed, and the washed to-be-sterilized objects are inputted into the sterilization chamber 11. At this time, the temperature of the to-be-sterilized objects are room temperatures at about 10˜20° C. The door 18 is closed, and the motor 35 is driven under the atmospheric environment, and the fan 34 is rotated. At the same time, the fan heater 24 is operated. At this time, as the air in the interior of the sterilization chamber 11 starts flowing and is heated by the fan heater 24 and is uniformly spread to the to-be-sterilized objects and in the sterilization chamber 11. The temperature of the sterilization chamber 11 is maintained at 50˜60° C. As the first sterilization chamber heater 25 and the second sterilization chamber heater 26 are operated along with the fan heater 24, the temperature of the sterilization chamber 11 can be more quickly raised.

In addition, the first sterilization can be implemented by means of the OH radicals in the middle of the above described processes. In a state that the temperature is raised, the violet ray generator 17 emits violet ray to the photo catalyst 36 coated on the fan 34, and with the air of reaction between the photo catalyst 36 and violet ray, the oxygen and moisture in the air can be converted, with the aid of the photo catalyst 36 and the violet rays, to a state of plasma in which oxygen ions such as singlet oxygen (01−), super oxide anion (02−), photo ozone (−03) and hydroxyl ion (OH−), hydrogen proton (H+) are mixed. In addition, it is circulated using the fan 34, and the generation and chain reaction are performed, thus generating a high density OH radical. So, the expensive plasma can be generated at a low cost in a simpler way. Here, the temperature of the sterilization chamber 11 can be adjusted to have a certain value within the above mentioned range. The first sterilization might be omitted if necessary depending on the process.

Next, when the hydrogen peroxide vapor supply adjusting valve 20 is open, the hydrogen peroxide vapor evaporated from the evaporator 13 can be supplied to the sterilization chamber 11, and the to-be-sterilized objects can be sterilized by means of the hydrogen peroxide vapor introduced into the sterilization chamber 11. At the time when hydrogen peroxide is supplied, the pressure of the sterilization chamber 11 is maintained at about 0.5˜1 Torr by opening the sterilization camber pressure adjusting valve 21. The pressure of the sterilization chamber 11 is preferably less than atmospheric pressure when the hydrogen peroxide vapor is injected, and it is preferably adjusted to a proper vacuum pressure below 100 Torr. When the hydrogen peroxide is inputted into the sterilization chamber 11, the fan 34 and the violet ray generator 17 do not work.

When hydrogen peroxide is injected, the pressure of the sterilization chamber 11 rises. When it becomes saturated, it keeps constant. In the above process, as the hydrogen peroxide vapor penetrates into the to-be-sterilized objects, the sterilization is performed. Afterward, the pressure of the sterilization chamber 11 rises by opening the sterilization chamber pressure release valve 22, so further penetrations of the hydrogen peroxide vapor are performed with respect to the to-be-sterilized objects, so that the second sterilization is performed to further sterilize the to-be-sterilized objects with hydrogen peroxide vapor

Under the atmospheric pressure, the violet ray lamp 39, the fan 34 and the fan heater 24 are operated, and a plasma state is obtained, thus generating OH radicals and performing the third sterilization. In the middle of the above operations, hydrogen peroxide is decomposed into water and oxygen, and in the middle of the generation of the OH radicals, the decomposition and deodorization of hydrogen peroxide are performed. Afterward, the sterilization chamber 11 is vacuumed for the purpose of eliminating the remaining hydrogen peroxide, and external air is supplied again, thus completing the sterilization process.

Since the sterilization chamber 11 and the to-be-sterilized objects are heated using the blower 16 and the fan heater 24, the phenomenon that hydrogen peroxide vapor is condensed is prevented, so more hydrogen peroxide vapor can penetrate into the to-be-sterilized objects with the aid of the above mentioned operations, thus enhancing the sterilization effects.

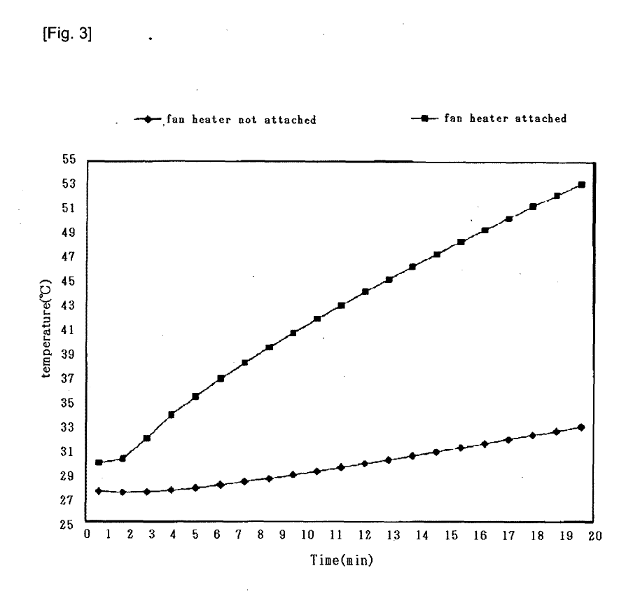

The graph of FIG. 3 shows a comparison of the temperature change of the sterilization chamber as time passes when the fan and the fan heater are not used and when they are used. As shown in FIG. 3, when the fan 34 and the fan heater 24 are used, it is possible to quickly raise the temperature of the sterilization chamber. Here, the volume of the sterilization chamber 11 used in the experiment is 50 liter, and the capacity of the blower 16 is 15˜20 m³/min, and the capacity of the fan heater 24 is 1500 W.

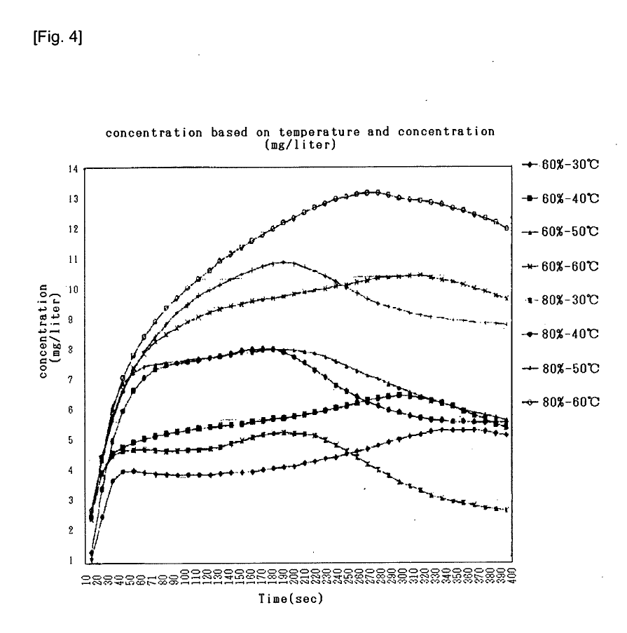

The graph of FIG. 4 shows a result of the measurement of the hydrogen peroxide concentration in the interior of the sterilization chamber based on the temperature of the sterilization chamber and the concentration of the hydrogen peroxide. As shown in FIG. 4, it seems that the concentrations of 60 wt % of 50° C. and 80 wt % of 40° C. are same, which shows that the rise of the temperature has as much effects as when the hydrogen peroxide with high concentration is used.

The sterilization apparatus 10 using hydrogen peroxide according to an embodiment of the present invention is directed to enhancing sterilization efficiencies by using the photo catalyst 36 coated on the fan 34 and the violet ray generator 17. The operations of the photo catalyst 36 and the violet rays are as follows.

In a state that the hydrogen peroxide is introduced into the interior of the sterilization chamber 11, when the violet ray generator 17 emits violet ray to the photo catalyst 36 coated on the fan 34, the oxygen and moisture and hydrogen peroxide in the air can be converted, with the aid of the photo catalyst 36 and the violet rays, to a state of plasma in which oxygen ions such as singlet oxygen (01−), super oxide anion (02−), photo ozone (−03) and hydroxyl ion (OH−), hydrogen proton (H+) are mixed. In addition, it is circulated using the fan 34, and the generation and chain reaction are performed, thus generating a high density OH radical. So, the expensive plasma can be generated at a low cost in a simpler way.

The oxidation power of OH radical is known to have sterilization power which is about 1.35 times higher than ozone, and about 1.86 times higher than chlorine dioxide and about 1.57 times higher than hydrogen peroxide. So, as compared to the sterilization method of using only hydrogen peroxide, it is possible to more enhance the sterilization power by using together the sterilization of using hydrogen peroxide and the plasma.

The following table 1 shows a result of the comparison experiment of the sterilization power when the fan, the fan heater and the violet ray generator do not operate and they are operated.

TABLE 1
(extinction rate/experiment rate)

| | fan, fan heater and | fan, fan heater and |
|---|---|---|
| | violet ray generator | violet ray generator |
| | do not operate | operate |
| Scissor for surgery | 5/10 | 10/10 |
| Hard Lumen | 0/10 | 9/10 |
| (φ1 mm × 600 mm) | | |
| Soft Lumen | 2/10 | 10/10 |
| (φ1 mm × 2,000 mm) | | |
| One side-closed PCD | 0/5 | 4/5 |
| (φ2 mm × 1,500 mm) | | |

In the present experiment, as a biological indicator system for checking the sterilization effects in the present experiments, 10⁶ number of Bacillus Stearothermophilus viable spore were used. As the to-be-sterilized objects accommodated in the sterilization chamber 11, 10 scissors for surgery in which living fungi were cultivated in a hinge which is hard to penetrate in and 10 hard lumen in which living fungi were cultivated at the center of φ1 mm×60 cm of a stainless pipe representing a hard endoscope, 10 soft lumen in which living fungi were cultivated at the center of φ1 mm×2 mm of the teflon pipe representing a soft endoscope and 5 PCDs in which a disc with a living fungus being cultivated is inserted into an end portion of an inner diameter of φ2×1,500 mm which is closed at its one side and is generally used as PCD (Process Challenge Device) of an oxide ethylene gas sterilizer and a vacuum type steam sterilizer were used, so the sterilization performances were compared and evaluated when the fan 34, the fan heater 24 and the violet ray generator 17 were operated and when they were not operated.

In the present experiment, the volume of the sterilization chamber 11 was 50 liter, the set temperature of the sterilization chamber 11 was 50° C., and the capacity of the blower 16 was 15˜20 m³/min, and the capacity of the fan heater 24 was 1500 W, and the concentration of the supplied hydrogen peroxide was 2 ml for 58 wt %, and the emitting level of the violet ray was 20 W, and the photo catalyst 36 as TiO₂, and the surface area of the photo catalyst 36 was about 40 cm².

The sterilization performance and the decomposition and deodorization effects of the hydrogen peroxide can be enhanced using the photo catalyst 36 and the violet ray. In the present invention, since the sterilization by plasma is performed in an atmospheric state, not in a vacuum state, the hydrogen peroxide can be decomposed in the principle below, and various smells occurring in the middle of sterilization and the inherent smell of the hydrogen peroxide can be decomposed and deodorized.

(1) As violet rays are emitted to the photo catalyst 36, electrons (e−) and positive holes (h+) generate.

(2) The generated electrons and positive holes react with oxygen, water and hydrogen peroxide and superoxide radical (02−) and hydroxy radical (OH+) are generated.

H₂O₂→H₂O+O₂

O₂+e−=O₂−(+)H₂O+h+=OH+

(3) The generated radicals (O₂−, OH++) have strong oxidation powder, thus oxidizing and reducing bad smell, virus, bacteria, fungus, NOx, VOCs.

(4) The above mentioned reactions are performed in chain and repeatedly using the fan 34, thus converting to CO₂ and H₂O.

By way of the above mentioned procedures, hydrogen peroxide is decomposed, and the inherent smell of hydrogen peroxide as well as various bad smells generating in the middle of sterilization can be decomposed and deodorized.

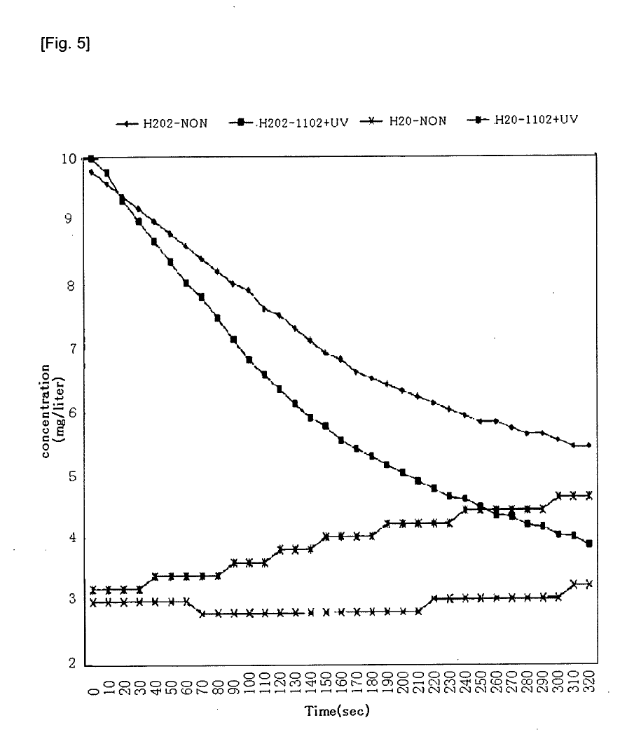

FIG. 5 is a graph showing the concentration changes of hydrogen peroxide measured every 10 seconds while comparing them before and after the fan and the violet ray generator are operated. As shown in FIG. 5, when comparing the concentrations of hydrogen peroxide and water of the sterilization chamber 11 when the photo catalyst 36 and the violet ray are used and when they are not used, the speed that the hydrogen peroxide becomes water is much faster when the photo catalyst and violet ray are scanned.

In the examples of the experiments, the set temperature of the sterilization chamber 11 was 50° C., and the capacity of the sterilization chamber 11 was 50 liter, and the emitting level of the violet ray was 20 W, and the coated surface area of the photo catalyst 36 was about 40 cm², and the capacity of the blower 16 was 15˜20 m³/min, and the supplied hydrogen peroxide was 2 ml with respect to the concentration 58 wt %.

The sterilization apparatus using hydrogen peroxide according to an embodiment of the present invention has a drying function of drying to-be-sterilized objects. Drying the to-be-sterilized objects is as follows.

When the sterilization process is finished, the temperatures of the to-be-sterilized objects and the remaining moistures in the interior of the sterilization chamber 11 are raised using the fan 34 and the fan heater 24 under atmospheric environment. At this time, the heating temperature is 30˜60° C. When the to-be-sterilized object is heated up to a proper temperature, the sterilization chamber pressure adjusting valve 21 is opened, and the vacuum pump 15 is operated, thus vacuuming, so the pressure of the sterilization chamber 11 is dropped to a certain level. At this time, the pressure of the sterilization chamber 11 is lower than atmospheric level, preferably lower than 100 Torr.

The sterilization chamber 11 is vacuum-exhausted for a certain time. If it is not fully vacuum-exhausted, the sterilization chamber pressure adjusting valve 21 is closed, and the sterilization chamber pressure release valve 22 is open, and air is injected again into the sterilization chamber 11. In addition, the fan 34 and the fan heater 24 are operated, and the temperature of the interior of the sterilization chamber 11 is raised, and then the vacuum exhaust process is performed. With the above mentioned heating and vacuum-exhaust processes are repeatedly performed, the moisture remaining in the to-be-sterilized objects can be fully eliminated.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A sterilization apparatus using hydrogen peroxide, comprising:
a housing which has a sterilization chamber for accommodating to-be-sterilized objects;
an evaporator connected with the sterilization chamber for supplying hydrogen peroxide vapor to the sterilization chamber for the purpose of sterilizing to-be-sterilized objects;
a hydrogen peroxide supply apparatus connected with the evaporator for the purpose of supplying liquid hydrogen peroxide to the evaporator;
a vacuum pump connected with the sterilization chamber for the purpose of providing a vacuum pressure to the sterilization chamber;
a blower which includes a fan disposed in the interior of the sterilization chamber for forcibly flowing the air of the sterilization chamber and a motor for driving the fan; and
is a fan heater disposed in the interior of the sterilization chamber for the purpose heating the air flowing by means of the fan,
wherein the fan is disposed closer to a side wall of one side of the housing, and the motor is disposed at an outer side of the side wall, and a rotary shaft of the motor passes through the side wall and is connected with the fan, and the fan heater is disposed between the fan and the side wall, and at the fan is installed a guide plate which faces the fan at the front side of the fan in the interior of the sterilization chamber and which rotates in a rotation direction that the air flowing to the fan gathers at the center of the fan, and the air of the interior of the sterilization chamber collides with the side wall by way of the fan and the fan heater and flows to the front side of the fan by way of the space between the guide plate and the inner surface of the housing.

2. A sterilization apparatus using hydrogen peroxide according to claim 1, further comprising a sterilization chamber temperature sensor disposed at the passage for detecting the temperature of the interior of the sterilization chamber.

3. A sterilization apparatus using hydrogen peroxide according to claim 1, further comprising:
a photo catalyst disposed in the interior of the sterilization chamber; and
is a violet ray generator which has a violet ray lamp disposed in the interior of the sterilization chamber for emitting violet ray to the photo catalyst.

4. A sterilization apparatus using hydrogen peroxide according to claim 3, wherein the photo catalyst is coated on at least one of the surface of the fan, the inner surface of the housing and the guide plate.

5. A sterilization apparatus using hydrogen peroxide according to claim 3, wherein the photo catalyst contains TiO₂.

6. A sterilization apparatus using hydrogen peroxide according to claim 1, further comprising:
a sterilization chamber pressure release valve connected with the sterilization chamber for introducing external air into the interior of the sterilization chamber.

7. A sterilization apparatus using hydrogen peroxide according to claim 1, further comprising:
a sterilization chamber heater which is engaged to the housing for the purpose of heating the housing and raising the internal temperature of the sterilization chamber.
